(19) United States
(12) Patent Application Publication
(10) Pub. No.: US 2016/0206300 A1
(43) Pub. Date: Jul. 21, 2016

(54) DEVICES, SYSTEMS AND METHODS FOR DISC AND MENISCUS REPAIR

(71) Applicant: Tenjin LLC, Brazoria, TX (US)

(72) Inventors: Christopher P. DOUGHERTY, Rogers, AR (US); Gary R. Heisler, Brazoria, TX (US); Robert A. Van WYK, St. Pete Beach, FL (US)

(73) Assignee: Tenjin LLC, Brazoria, TX (US)

(21) Appl. No.: 14/601,789

(22) Filed: Jan. 21, 2015

(51) Int. Cl. A61B 17/00

(52) U.S. Cl. CPC A61B 17/00491; A61B 2017/00495; A61B 17/00234

(57) ABSTRACT

Arising from the discovery that a non-immunogenic poly-synthetic bioadhesive based on polyphenolic proteins produced by sandcastle worms (Phragmatopoma californica) may be used to anneal tears of the meniscus and annulus fibroses and repair spinal disc herniations, herein disclosed are suture-less meniscal and disc repair devices, systems and methods that may be implemented least invasively, in fluid filled or dry/semi-dry environments using simple instrumentation and without the need for suturing or the placement of anchors. The present invention is less technically demanding for the practioner and thus expected enhance outcomes and represent a distinct advancement over prior art techniques presently available.

### PRIORITY

This application claims the benefit of U.S. Provisional Application No. 61/965,062 filed Jan. 21, 2014, the contents of which are hereby incorporated by reference in their entirety.

### FIELD OF THE INVENTION

The present invention relates to devices, systems and techniques for the repair of meniscal tears, spinal disc herniations, and annulus tears. More particularly, the invention relates to an application device assembly uniquely configured for the delivery of a suitable two-part bioadhesive to a remote tissue tear in the context of meniscal and spinal disc repair procedures.

### BACKGROUND OF THE INVENTION

The repair of meniscal tears, spinal disc herniations, and annulus tears is known in the art to be problematic for the surgeon. Repairs of this type are generally performed using minimally invasive surgical techniques, techniques that, by definition and design, operate with less injury to the body than open surgery but, on the flip side, afford only limited access to the target surgical site. Current repair methods for meniscal tears use implant devices, frequently in combination with one or more sutures. Illustrative examples of such devices presently available for commercial use include the Sequent™ Meniscal Repair Device by Conmed, Inc. (Utica, N.Y.), the Fast-Fix™ Device by Smith and Nephew, Inc. (Andover, Mass.) and the Meniscal Cinch™ by Arthrex, Inc. (Naples, Fla.). While these devices and methods represent significant advancements in the art over previous meniscal repair techniques, disadvantages and deficiencies nevertheless remain that limit their application and affect patient outcomes. Among these is the need for the surgeon to place fixation devices through the body of the meniscus through healthy tissue and deposit either a suture construct or hard implant outside the capsule. Such implants require specific and precise placement through the capsule as failure to place them adequately through the tissue can result in dislodgement of the device(s) into the intra-articular space. Furthermore, these and other similar devices function by compression of the meniscus tear via suture(s) that pass through either ends of these devices. These sutures can become entangled or prematurely knotted, thereby resulting in functionally inadequate fixation at the repair site. In addition, conventional devices of the prior art can prematurely deploy from their insertion handle into the joint, which, in turn, gives rise to implant failure. Finally, such devices may also fail by deploying only one of their anchor bodies through the capsule, while the other remains inside the device, which, again results in failure of the implant. Similarly, the treatment of spinal disc herniation and annulus tears is known in the art to be quite challenging for the surgeon. Currently available treatment options include, for example, the Octopus Spinal Annular Repair System (OSA) by NewVert, Inc. (Netanya, ISREAL), and the Inclose Surgical Mesh System and Xclose Tissue Repair System by Anulex Technologies, Inc. (Minnetonka, Minn.) and the Barricade® annular repair device, by Intrinsic Therapeutics, Inc. (Woburn, Mass.). While these devices and methods represent significant advancements in the art over previous spinal disc repair techniques, disadvantages and deficiencies again remain that limit their application and impact patient outcomes. For example, such devices are predicated upon placing barrier type containment devices into the annular defect and deploying on the inside of the disc to serve as a barrier to prevent further extrusion of the nucleus pulposis from the disc. Accordingly, they can fail to deploy on insertion. In addition, smaller annular tears require the tear to be enlarged to allow insertion of the repair device. Other devices utilize anchors and suture passing between the anchors to provide a method for annulus closure that is very similar to the aforementioned meniscal repair protocol and thus is likewise subject to the aforementioned modes of failure.

Accordingly, there remains a need in the art for devices, systems and methods for repair of meniscal tears, spinal disc herniation, and annulus tears that are less technically challenging for the surgeon and thus can afford better patient outcomes.

### SUMMARY OF THE INVENTION

Given the above-described need in the art, it is a goal of the present invention to provide a method for meniscal and spinal disc repair that may be implemented in the context of a minimally invasive surgical procedure.

An additional objective of the present invention is to provide a method for meniscal and spinal disc repair that may be used in either a fluid filled or semi-dry environment.

Yet another objective of the present invention is to provide a method for meniscal and spinal disc repair that utilizes simplified instrumentation.

A further objective of the present invention is to provide a method for meniscal and spinal disc repair that does not require highly developed surgical skills to operate.

A further objective of the present invention is to provide a method for meniscal and spinal disc repair that does not require suture or suture anchors.

Finally, a further objective of the present invention is to provide an assembled application device and system for meniscal and spinal disc repair that allows for the minimally invasive application of a two-part adhesive with a short set time.

It will be understood by those skilled in the art that one or more aspects and embodiments of the present invention can meet certain of the afore-mentioned objectives, while other aspects and embodiments can meet certain other objectives. Each objective may not apply equally, in all its respects, to every aspect of this invention. As such, the foregoing objectives can be viewed in the alternative with respect to any one of the aspects and embodiments of the present invention as follows:

Certain marine animals, such as mussels of genus Mytilus and worms of the genus Phragmatopoma, are known to produce polyphenolic proteins that, when combined in an aqueous environment, quickly set up as a bioadhesive (e.g., initially set within thirty seconds and fully set in one hour). Researchers at the University of Utah have isolated and replicated illustrative embodiments of such proteins and created a poly-synthetic bioadhesive suitable for use in humans. (See Wang, C. S, Svendsen, K. K., and Stewart, R. J. (2010), “The adhesive system of the Sandcastle worm, in Adhesion Phenomenon in Nature”, ed. J. Byern and I. Grunwald, Springer). Critically, the adhesive is non-immunogenic and therefore does not elicit an immune response; as such, the bioadhesive, and any construct or component fabricated or formed therefrom, may remain in situ over an extended period without giving rise to a negative immune consequence such as inflammation and rejection. While such bioadhesive proteins have to date found utility in the context of heart muscle repair (see Lang, Nora et al., “A Blood-Resistant Surgical Glue for Minimally Invasive Repair of Vessels and Heart Defects”, Science Translational Medicine (January 2014), Vol. 6, Issue 218, p. 218ra6), the present invention represents the first novel use of polyphenolic bioadhesive proteins to anneal tears of the meniscus and annulus fibrosis. In this manner, the present invention avoids the shortcomings of prior art devices and methods that rely upon anchor bodies, sutures, mesh or expanding bodies to accomplish repair. While characteristics such as set and cure times may vary based depending on the precise composition of the selected poly-synthetic bioadhesive, the bioadhesives utilized in the context of the present invention function in a fashion analogous to the above-described “sandcastle worm glue”.

Accordingly, one aspect of the present invention relates to an enhanced method for repair of meniscal tears, spinal disc herniations, and annulus tears in which tissue is bonded using an advanced poly-synthetic bioadhesive that is both non-immunogenic and equally suited to fluid filled and semi-dry environments.

Another aspect of the present invention relates to an assembled application device and system for the repair of meniscal tears, spinal disc herniations, and annulus tears, wherein the system includes the afore-mentioned two-part poly-synthetic bioadhesive that (a) may be used in a fluid filled or semi-dry environment and (b) is not attacked by the body's immune system, and the assembled application device is configured to administer the bioadhesive in the context of a minimally invasive surgical procedure in which access to the repair site is limited. As noted above, since the preferred bioadhesive is non-immunogenic, it will not elicit an immune response. Accordingly, constructs of the present invention may remain in situ indefinitely without giving rise to inflammation or other immunorejection consequence.

Yet another aspect of the present invention relates to a novel application device assembly uniquely configured to deliver the polyphenolic protein components of the afore-mentioned two-part poly-synthetic bioadhesive to a remote repair site and critically prevent the mixing of the respective parts until delivery at the site desired for repair. In a preferred embodiment, the device of the present invention is designed to function as a single-use, sterile-processed disposable applicator. An illustrative embodiment of a preferred application device of the present invention, one particularly configured for the delivery of a two-part poly-synthetic bioadhesive to a remote surgical site of interest, comprises an assembly of the following components:
i. a tubular applicator body having open proximal and distal ends and first and second adjacent parallel lumens extending therebetween configured to receive first and second components of a two part poly-synethetic bioadhesive;
ii. an elongate tubular section extending from the distal end of the tubular applicator body that includes parallel first and second elongate tubes encased by an elongate polymeric sheath and having open proximal and distal ends, wherein the proximal end of the first tube is attached to and in exclusive fluid communication with the first lumen and the proximal end of the second tube is attached to and in exclusive fluid communication with the second lumen, respectively;
iii. a mixing element including (i) a tubular proximal portion having adjacent parallel first and second chambers, wherein the first chamber is configured to attach to the distal end and be in exclusive fluid communication with the first elongate tube and the second chamber is configured to attach to the distal end and be in exclusive fluid communication with the second elongate tube, and (ii) a distal portion in fluid communication with both the first and second chambers that includes a helical arm configured for the mixing the first and second bioadhesive components upon flow from the first and second lumen, into and through the first and second elongate tubes and the first and second chambers, and into the distal portion of the mixing element;
iv. a nozzle element including a proximal portion configured to receive the mixing element and attach to the distal end of the elongate tubular section and a distal portion for dispensing mixed material to the remote surgical site of interest; and
v. a plunging element including a proximal flange portion and first and second distally extending elongate members, wherein the first elongate member is sized and positioned to be slidably received by the first lumen and the second elongate member is sized and positioned to be received by second first lumen, wherein the plunging element serves to displace the first and second bioadhesive components from the first and second lumen, into and through the first and second elongate tubes and the first and second chambers, and eject the mixed material from the distal portion of the nozzle element to the remote surgical site of interest.

Yet a further aspect of the present invention relates to a sterile kit containing the above-described application device pre-loaded with the requisite polyphenolic protein components of the two-part synthetic bioadhesive, as well as supplemental and/or secondary device components necessary for completing repairs at multiple sites, or when the repair at a single site is interrupted.

These and other aspects of the present invention are described herein below with reference to a number of specific embodiments. However, it is to be understood that both the foregoing summary of the invention and the following detailed description are of a preferred embodiment, and not restrictive of the invention or other alternate embodiments of the invention. Further objects and features of the invention will become more fully apparent when the following detailed description is read in conjunction with the accompanying figures and examples.

### BRIEF DESCRIPTION OF THE DRAWINGS

Various aspects and applications of the present invention will become apparent to the skilled artisan upon consideration of the brief description of figures and the detailed description of the present invention and its preferred embodiments that follows:

FIG. 1 depicts a plan view of the body component of an application device of the present invention configured for the application of a two-part adhesive in accordance with the principles of this invention.

FIG. 2 is a side elevational view of the objects of FIG. 1.

FIG. 3 is a side elevational sectional view of the objects of FIG. 1 at location A-A of FIG. 1.

FIG. 4A is an expanded view of the mid-portion of FIG. 3 at location A of FIG. 3.

FIG. 4B is an expanded view of the distal portion of FIG. 3 at location B of FIG. 3.

FIG. 5 is a perspective exploded assembly view of an application device configured for application of a two-part adhesive in accordance with the principles of this invention, including the body component of FIG. 1.

FIG. 6 is an expanded perspective view of the distal portion of the objects of FIG. 5.

FIG. 7 is a side elevational view of an assembled application device configured for application of a two-part adhesive in accordance with the principles of this invention.

FIG. 8 is a perspective view of the objects of FIG. 7.

FIG. 9 depicts an assembled application device in use repairing a meniscal tear in accordance with the principles of this invention.

FIG. 10 depicts an assembled application device in use repairing a disc herniation in accordance with the principles of this invention.

### DETAILED DESCRIPTION OF THE PREFERRED EMBODIMENTS

Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims. Accordingly, unless otherwise defined, all technical and scientific terms used herein have the same meaning as commonly understood by one of ordinary skill in the art to which the present invention belongs. However, in case of conflict, the present specification, including definitions below, will control.

### Elements of the Present Invention:

In the context of the present invention, the following definitions apply:

The words “a”, “an” and “the” as used herein mean “at least one” unless otherwise specifically indicated. Thus, for example, reference to an “opening” is a reference to one or more openings and equivalents thereof known to those skilled in the art, and so forth.

The term “proximal” as used herein refers to that end or portion which is situated closest to the user of the device, farthest away from the target surgical site. In the context of the present invention, the proximal end of the application device includes the proximal flange.

The term “distal” as used herein refers to that end or portion situated farthest away from the user of the device, closest to the target surgical site. In the context of the present invention, the distal end of the application device of the present invention includes the demountable nozzle portion.

In the context of the present invention, the term “cannula” is used to generically refer to the family of elongate lumened surgical instruments that facilitate access across tissue to an internally located surgery site.

The terms “tube” and “tubular” are interchangeably used herein to refer to a generally round, long, hollow component having at least one central opening often referred to as a “lumen”.

The term elongate is used herein, particularly in reference to the distal portion of the body of the application device (or “applicator”), to refer to an element that is unusually long in relation to its width.

The present invention makes reference to certain “flange” portions. In the context of the present invention, the term flange refers to a projecting, preferably flat, rim, collar, or rib on an object, serving to strengthen one component or attach one component to another.

The terms “lengthwise” and “axial” as used interchangeably herein to refer to the direction relating to or parallel with the longitudinal axis of a device. The term “transverse” as used herein refers to the direction lying or extending across or perpendicular to the longitudinal axis of a device.

The term “lateral” pertains to the side and, as used herein, refers to motion, movement, or materials that are situated at, proceeding from, or directed to a side of a device.

The term “medial” pertains to the middle, and as used herein, refers to motion, movement or materials that are situated in the middle, in particular situated near the median plane or the midline of the device or subset component thereof.

The term “rotational” as used herein refers to the revolutionary movement about the center point or longitudinal axis of the device. In the context of the present invention, the nozzle and mixing components are mounted to and demounted from the distal end of the applicator body through relative rotation, which, in turn, causes mating locking elements to be alternatively engaged or disengaged.

The present invention finds utility in the repair of tears in body tissues, particularly meniscal tears, spinal disc herniations, and annulus tears. As used herein, the term “tissue” refers to biological tissues, generally defined as a collection of interconnected cells that perform a similar function within an organism. Four basic types of tissue are found in the bodies of all animals, including the human body and lower multicellular organisms such as insects, including epithelium, connective tissue, muscle tissue, and nervous tissue. These tissues make up all the organs, structures and other body contents. Although the present invention is described in detail in connection with tissues of the knee and spinal column, it should not be construed as limited thereto; rather, the present invention may have broad application in connection with the suture-less repair of other types of tissues, such as arthroscopic and musculoskeletal tissues.

The term “bioadhesive” as used herein refers to an adhesive substance produced by or obtained from living organisms, or used on living tissue. In the context of the present invention, the bioadhesive preferably comprises two parts, two distinct materials that, when mixed, form a singular bioadhesive material in a manner akin to the conventional two-part epoxy. Illustrative examples of such two-part bioadhesives contemplated by the present invention are described in U.S. Pat. Nos. 5,015,677, 6,506,577, and 7,186,908, the contents of which are incorporated by reference herein.

Preferred bioadhesives arise from the cross-linking, of naturally occurring polyphenolic proteins, such as those expressed by several mussel species of the genus Mytilus or worms of the genus Phragmatopoma (discussed in greater detail below), or their synthetic equivalents. Such bioadhesive polyphenolic proteins exhibit excellent adhesive properties on a variety of surfaces, particularly surfaces submerged in water. In the context of the present invention, the “polyphenolic proteins” preferably contain one or more, preferably 10 to about 400, more preferably from about 50 to about 150 units of a repeating decapeptide such as described in U.S. Pat. No. 5,015,677. Suitable decapeptides may be obtained by the method described by Waite in Journal of Biological Chemistry 258, 2911-15 (1983). and U.S. Pat. No. 4,585,585. Alternatively, the bioadhesive polyphenolic proteins may be obtained through biochemical synthesis or genetic engineering techniques well known to those skilled in the art.

In the Summary above and the Examples below, the present invention makes reference to a specific bioadhesive derived from the Sandcastle worm (Phragmatopoma californica) that is referred to herein as “worm glue”. Other species that produce underwater glues that may find utility in the context of the present invention include certain species of mussels, oysters, barnacles and caddisfly larvae. See Rebecca A. Jensen and Daniel E. Morse in “The bioadhesive of Phragmatopoma californica tubes: a silk-like cement containing L-DOPA” (1988), Journal of Comparative Physiology B, vol. 158, Issue 3, pp 317-324, and Rzepecki, L. M et al. in “Molecular diversity of marine glues: Polyphenolic proteins from five mussel species” (1991), Molecular Marine Biology and Biotechnology, vol. 1 (1): 78-88.

In 2005, researchers from the University of California, Santa Barbara (UCSB) discovered that the glue used by the Sandcastle worm to build its protecting tube was made of specific proteins with opposite charges, proteins referred to as “polyphenolic proteins” that find utility as bioadhesives. Since then synthetic equivalents have been generated and alleged to have utility as an immunogenic biocompatible medical adhesive (or “bioadhesive”). See http://en.wikipedia.org/wiki/Sandcastle_worm as well as Henry Fountain's Apr. 12, 2010 article in the New York Times entitled “Studying Sea Life for a Glue That Mends People”.

The polyphenolic proteins that are the basis of any such bioadhesive preferably contain side chains of phosphate and amine groups, both of which are well-known adhesion promoters that likely assist in wetting the substrate surface. Like an epoxy, the preferred bioadhesive of the instant invention results from the mixing of two distinct polyphenolic proteins, each with different proteins and side groups. The adhesive generally sets in about 30 seconds and, as the proteins cross-link, cures to a consistency of shoe leather in about six hours.

Bioadhesive polyphenolic proteins can impart water-compatible characteristics to any adhesive formulation through their increased monomeric molecular weight, reduced tendency to diffuse from the application site, and increased number and variety of reactive residues, such as the “phenol-like” residues tyrosine and DOPA, that are especially capable of displacing water. Molecular weight plays a key role in the strength of the resulting bioadhesive: if the molecular weight is too low, the curing will not produce sufficiently strong bonds and accordingly, the adhesive will not be able to bind two substrates together. On the other hand, if the molecular weight is too high, such as when the bioadhesive contains too many (e.g., above 400) repeating monomers, the resulting bioadhesive will be too viscous for practical application. Preferably, the bioadhesive polyphenolic proteins comprise mixtures containing about 50 to about 150 repeating units so as to maximize the adhesive strength of the protein while still maintaining sufficient fluidity for easy handling.

Bioadhesives of the present invention may optionally include other proteinaceous units, chain extenders, fillers and/or cross-linking promoters and preferably exhibit an adhesive strength after curing of at least about 100 gm/cm² when used on soft, tissue, preferably at least about 150 gm/cm², which indicates that a sufficient amount of intermolecular bonds and bonding between the substrate and the adhesive has been achieved to adhere the substrate to the adhesive.

Existing medical grade bioadhesives are highly immunogenic, with initial animal experiments with new synthetic showing no immune response. However, inside the body, it is preferable for the bioadhesive to eventually degrade, ideally at roughly the same rate as the bone or tissue regrows. Accordingly, in the context of the present invention, the synthetic bioadhesive is preferably biodegradable, including proteins that are broken down by specialized cells.

In the Examples below, the present invention makes reference to various fastener pair mechanisms that serve to establish and secure the arrangement of various device components, such as the nozzle and mixing element to the distal end of the applicator body. It will be readily understood by the skilled artisan that the position of the respective coordinating elements (e.g., mating slots and protrusions) may be exchanged and/or reversed as needed. Alternate fastener embodiments, such mating screw threads, are also contemplated.

The instant invention has both human medical and veterinary applications. Accordingly, the terms “subject” and “patient” are used interchangeably herein to refer to the person or animal being treated or examined. Exemplary animals include house pets, farm animals, and zoo animals. In a preferred embodiment, the subject is a mammal.

### Utilities of the Present Invention:

As noted above, certain disadvantages and deficiencies plague present day devices and techniques for the repair of meniscal tears, spinal disc herniations, and annulus tears. For example, at present, all commercially available annulus repair devices are suture based, require meticulous technique and accordingly are generally successful only in the hands of a highly skilled surgeon. The present invention arises from the discovery that a non-immunogenic poly-synthetic bioadhesive, such as that based on polyphenolic proteins produced by sandcastle worms (Phragmatopoma californica), may be used to anneal tears of the meniscus and annulus fibroses and repair spinal disc herniations. Accordingly, the devices, systems and methods of the present invention improve upon presently available techniques by providing a suture-less system that may be applied through a minimally invasive technique. As it avoids the requirement for knot tying, the present invention does not require the surgeon skill set.

In particular, the present invention allows the practioner to introduce a repair device into the joint without the need to pass a corresponding suture and further allows the repair to be carried out in a truly “all inside” technique, whereby no suture needs to be passed, and fixation is not predicated upon penetration of a capsule by a fixation device. What is unique about the method of the present invention is that fixation is carried out at the site of repair, in contrast to repairs that are dependent upon anchoring sites outside the joint in which fixation is entirely dependent upon the suture passing between the anchor points.

Additionally, the devices, systems and methods of the present invention are unique in that they provide for direct repair at the site of the tear, as opposed to compression at the site of the tear with fixation outside the joint. A critical distinction from the prior art is that the devices, systems and methods of the present invention allow for intra-articular repair at the site of the tear as opposed to anchor points outside the capsule. In this manner, the present invention provides the surgeon with the ability to repair tears in multiple planes. The devices, systems and methods of the present invention are further unique in their ability to provide for a mechanical sealing of free edge tears with a pliability that does not affect the overall function of the meniscus following repair.

The devices, systems, and methods of the present invention are unique for repair of annulus tears of the spine in that they provide for the only true suture-less sealing of tears of the annulus. The novelty and distinctness of this repair style allows both the skilled practitioner and the non-skilled practitioner to complete the same repair simply with the aid of specifically designed repair devices.

These devices, systems, and methods of the present invention provide to the surgeon the ability to maintain the bioadhesive components in two (or more) separate chambers and to not allow the components to mix and thus the glue to cure until applied at the site of the desired repair. More particularly, the poly-synthetic bioadhesive is prepared on site, on demand, in a specific concentration and the delivery is ensured by the different lumens and applied directly at the site of desired repair. The tissue at the repair site is preferably held in the desired compressed position by the delivery tube and insertion device until the biosynthetic adhesive has set.

In preferred embodiments, the biosynthetic adhesive sets in a short time, approximately thirty seconds in presently available formulations, though other formulations with possibly longer set times are anticipated. The cure time of presently available formulations may be as little as one hour or as much as six hours. When fully cured the adhesive has the consistency of leather, which is ideal for the repair of meniscal tears, disc herniations and annulus fibroses in accordance with the principles of this invention. However, alternative adhesive formulations that are non-immunogenic and/or biodegradable, that may be applied in wet and/or dry/semi-dry environments, and that have suitable set and cure times are contemplated and may fall within the scope of this invention.

In conjunction with the two-part poly-synthetic bioadhesive, the present invention contemplates a unique application device for least-invasive meniscus or disc repairs, preferably in the form of a sterile kit that includes the requisite bioadhesive components pre-loaded into the application device. The kit may further include substitute and/or supplemental mixing and application elements, the functions of which will become apparent in the following explanation.

The injection of a two-part adhesive (such as the poly-synthetic bioadhesive contemplated by the present invention) with a short set time can be problematic, particularly in demanding applications when thorough mixing of components with tightly held mix ratios is required. For example, one must take precaution to ensure that voids are not present in the material path proximal to the mixing site. In addition, when materials are mixed, they must be applied in a timely manner since flow at an insufficient rate or pauses in the flow can lead to partial or complete setting of the adhesive in the mixing portion and/or in portions of the path distal to the mixing portion. If the flow is interrupted and setting of the material in these regions occurs, the injection system must allow easy replacement of either or both of the mixing portion and the distal flow path. When such an injection device is applied in relatively inaccessible sites, as in minimally invasive surgical applications, the flow path of the components may have significant length so that mixing of the components must be accomplished at the distal end of the device. The uniquely designed application device of the present invention addresses the foregoing needs and thus represents a significant improvement in the art of suture-less tissue repair.

### ILLUSTRATIVE EMBODIMENTS OF THE PRESENT INVENTION

Hereinafter, the present invention is described in more detail by reference to the exemplary embodiments. However, the following examples only illustrate aspects of the invention and in no way are intended to limit the scope of the present invention. As such, embodiments similar or equivalent to those described herein can be used in the practice or testing of the present invention.

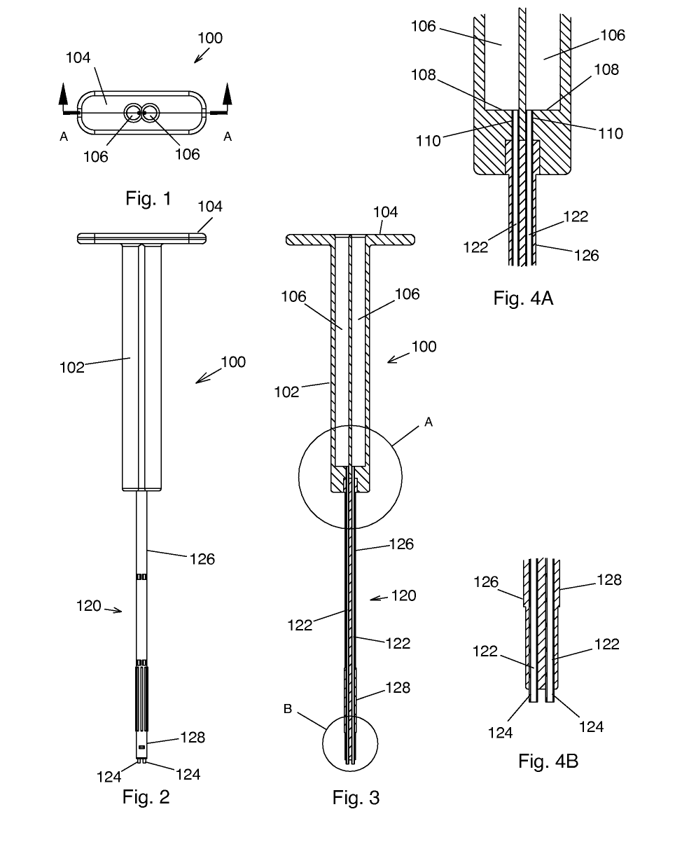

FIGS. 1 through 4 depict the body 100 of an application device for a two-part poly-synthetic bioadhesive according to the principles of this invention. Body 100 has a proximal portion 102 with a flange 104 at its proximal end, and lumens 106 extending to closed distal ends 108 in communications with lumens 110. Elongate distal portion 120 has two distally extending preferably metallic tubes 122 surrounded and encased by polymeric member 126. Metallic tubes 122 at their proximal end are coaxial with lumens 110 of proximal portion 102 and are positioned therein. Distal ends 124 of metallic tubes 122 protrude from distal end 128 of polymeric member 126. Metallic tubes 122 provide a flow path for materials placed in lumens 106.

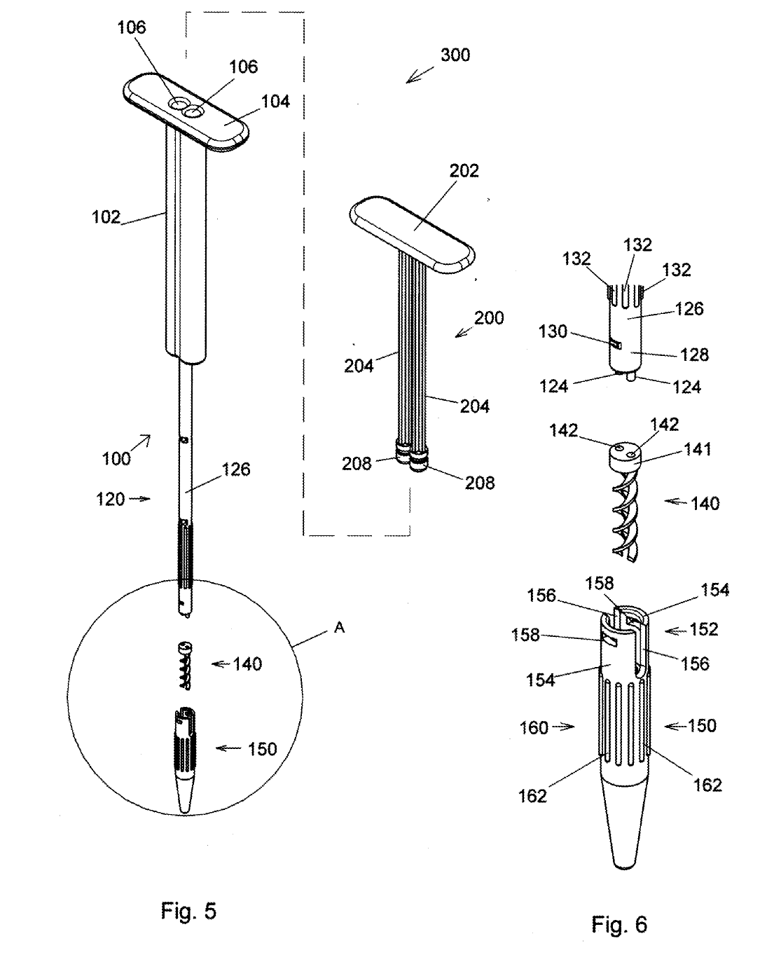

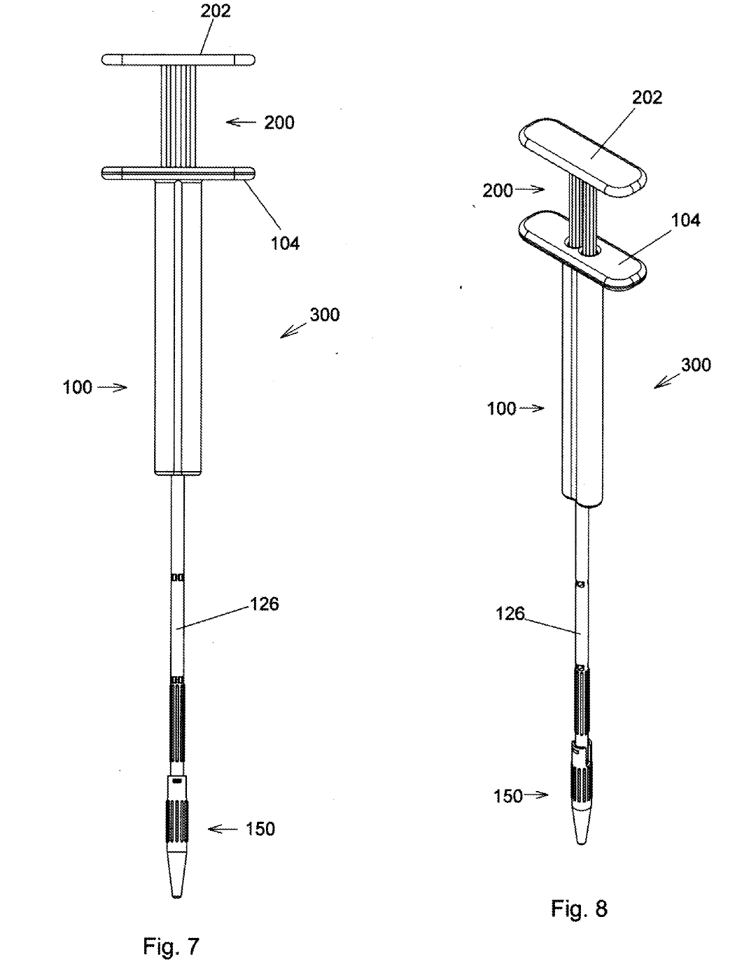

Referring now to FIGS. 5 through 8, which depict the assembly of an application device 300 for a two-part poly-synthetic bioadhesive in accordance with the principles of this invention, as best seen in FIG. 6, near distal end 128 of polymeric member 126 are formed thereon flanges 130 having tapered distal and lateral surfaces. Proximal to flanges 130, ridges 132 are formed on member 126 so as to provide a gripping surface. Mixing element 140 has a cylindrical proximal portion 141 having lumens 142 sized and positioned to receive distal ends 124 of metallic tubes 122, and a helically formed distal portion configured for the mixing of materials flowing from the distal ends 124 of tubes 122. Nozzle 150 has a central lumen having a proximal portion configured to receive mixing element 140 and a distal portion for dispensing mixed material. Proximal portion 152 of nozzle 150 is formed of proximally extending portions 154 separated by slots 156, portions 154 having formed therein openings 158 configured such that when distal end 128 of polymeric member 126 is inserted into nozzle 150 flanges 130 of member 126 and openings 158 of nozzle 150 together form a fastener pair to prevent demounting of nozzle 150. Mid-portion 160 of nozzle 150 has ridges 162 formed thereon so as to provide a gripping surface. Nozzle 150 may be demounted from distal end 128 of polymeric member 126 by rotating nozzle 150 such that proximal portions 154 of nozzle 150 spread apart and disengage flanges 130 of member 126. Plunging element 200 has a proximal flange 202 portion and distally extending elongate members 204 sized and positioned to be received by lumens 106 of body 100. Elongate members 204 have at their distal ends 206 sealing elements 208.

In a preferred embodiment, polymeric element 126 is made from a material that allows distal portion 120 of body 100 to be bent at use to allow improved access to a remote site of interest.

In a preferred embodiment, application device is supplied as part of a sterile kit containing application device 300 pre-loaded with the two adhesive components, wherein distal end 128 of distal element 126 is preliminarily sealed with a cap that may be discarded prior to use. In use, following removal of the cap, device 300 is positioned with the distal end pointing upward and plunging element 200 advanced distally until an uninterrupted flow of each material from the respective distal ends 124 of tubes 122 is observed. Mixing element 140 is then mounted to the distal end 128 of element 126, and the assembly is inserted distally into nozzle 150 such that flanges 130 of element 126 engage openings 158 of nozzle 150. Device 300 is now ready for use.

Setting of the poly-synthetic adhesive begins upon mixing of the components. It is, therefore, necessary that the adhesive be applied in a manner that ensures that substantially unset adhesive reaches the repair site. Interruptions in the application may cause partial or complete setting of the adhesive in the nozzle leading to suboptimal properties in the dispensed material. Accordingly, if an interruption occurs in the application process, it may be necessary to remove and replace nozzle 150 and mixer 140. This may be accomplished in the following manner: nozzle 150 is rotated until flanges 130 of element 126 are disengaged from the openings 158 of nozzle 150 and the nozzle is removed distally from distal end 128 of element 126. Mixing element 140 is then removed. Subsequently, the distal end of device 300 is elevated and element 200 is advanced distally until uninterrupted flow of both components is again observed. Thereafter, a new mixing element 140 and nozzle 150 (supplied as part of the sterile kit) may be installed in the manner previously described in preparation for continued use. In a preferred embodiment, multiple nozzles 150 and mixing elements 140 are supplied in the kit.

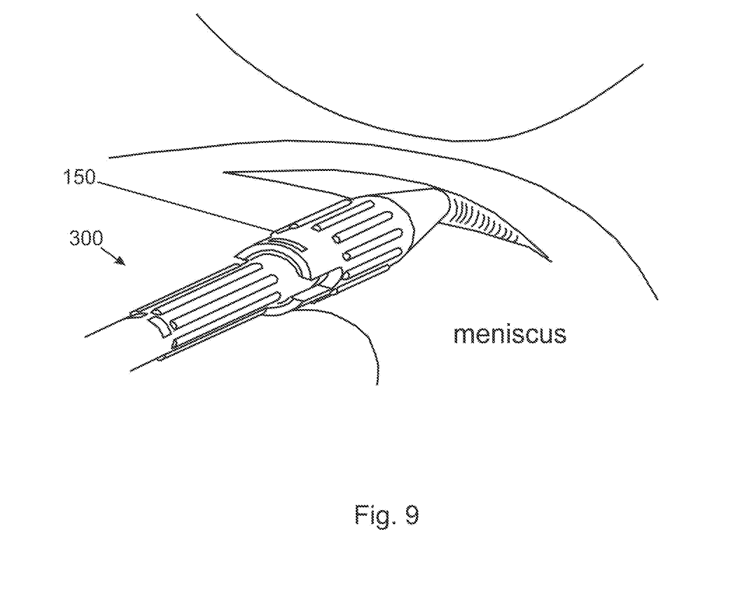

FIG. 9 shows application device 300 in use repairing a meniscal tear in a fluid-filled environment in a minimally-invasive fashion according to the method of this invention. The repair is done in the following manner: Device 300 is prepared according to the procedure previously herein described, device 300 having been furnished in a sterile kit pre-loaded with the two poly-synthetic adhesive components. When air has been expelled from the system in the manner previously herein described and mixing element 140 and nozzle 150 have been mounted to device distal portion 126 of body 100 of device 300 as previously described, nozzle 150 is positioned relative to the repair site so as to allow proper application of the adhesive, the components of which are mixed by mixing element 140 and directed by nozzle 150 in a manner which allows proper positioning and closing of the tear prior to setting of the adhesive. The tissue at the repair site is preferably held in the desired compressed position by the nozzle of the application device and/or such other instruments as the surgeon may deem appropriate and effective until the adhesive has set, preferably on the order of thirty seconds. If application of the adhesive is interrupted, nozzle 150 and mixing element 140 may be removed and replaced using the process previously herein described and the repair may then be completed.

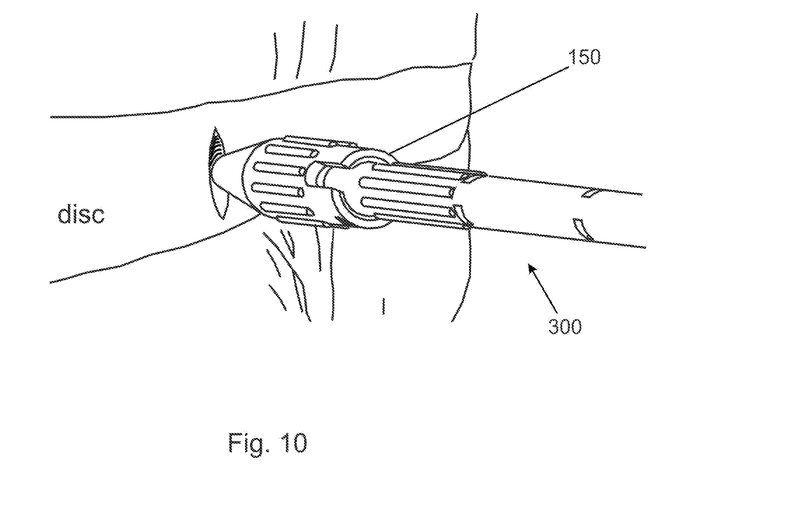

FIG. 10 depicts the minimally invasive repair of a disc herniation in a dry/semi-dry environment using device 300 according to the method of this invention. The steps of the repair procedure are analogous to those of the meniscal repair described above.

### INDUSTRIAL APPLICABILITY

As noted previously, the present invention arose from the discovery that a non-immunogenic poly-synthetic bioadhesive, such as that based on polyphenolic proteins produced by sandcastle worms (Phragmatopoma californica), may be used to anneal tears of the meniscus and annulus fibroses and repair spinal disc herniations. The suture-less meniscal and disc repair devices, systems and methods of the present invention utilize a poly-synthetic bioadhesive based on or analogous to the above-described “worm glue” and thus represent an advancement over currently used techniques that will result in improved patient outcomes and is less demanding technically for the surgeon. The invention may be implemented least invasively, in fluid filled or dry/semi-dry environments, using simple instrumentation. Suturing or the placement of anchors is not required. Although the present invention is described in detail with respect to the sandcastle worm glue, it will be readily apparent to the skilled artisan that the utility of the present invention extends to alternative adhesive formulations that are non-immunogenic but may yet be applied in wet or dry/semi-dry environments and have suitable set and cure times are contemplated.

The disclosure of each publication, patent or patent application mentioned in this specification is specifically incorporated by reference herein in its entirety. However, nothing herein is to be construed as an admission that the invention is not entitled to antedate such disclosure by virtue of prior invention.

The invention has been illustrated by reference to specific examples and preferred embodiments. However, it should be understood that the invention is intended not to be limited by the foregoing description, but to be defined by the appended claims and their equivalents.

What is claimed:

1. An application assembly for delivering a two-part poly-synthetic bioadhesive to a remote surgical site of interest, said device comprising:
i. a tubular applicator body having open proximal and distal ends and first and second adjacent parallel lumens extending therebetween, wherein said first and second lumens are configured to receive first and second components of said two part poly-synethetic bioadhesive;
ii. an elongate tubular section extending from the distal end of said tubular applicator body, wherein said elongate tubular section comprises adjacent parallel first and second elongate tubes encased by an elongate polymeric sheath and having open proximal and distal ends, further wherein the proximal end of said first tube is attached to and in exclusive fluid communication with said first lumen and the proximal end of said second tube is attached to and in exclusive fluid communication with said second lumen, respectively;
iii. a mixing element comprising (i) a tubular proximal portion having adjacent parallel first and second chambers, wherein said first chamber is configured to attach to the distal end and be in exclusive fluid communication with said first elongate tube and said second chamber is configured to attach to the distal end and be in exclusive fluid communication with said second elongate tube, and (ii) a distal portion in fluid communication with both said first and second chambers that includes a helical arm configured for the mixing said first and second bioadhesive components upon flow from said first and second lumen, into and through said first and second elongate tubes and said first and second chambers, and into said distal portion of the mixing element;
iv. a nozzle element comprising a proximal portion configured to receive said mixing element and attach to the distal end of said elongate tubular section and a distal portion for dispensing mixed material to said remote surgical site of interest; and
v. a plunging element comprising a proximal flange portion and first and second distally extending elongate members, wherein said first elongate member is sized and positioned to be slidably received by said first lumen and said second elongate member is sized and positioned to be received by second first lumen, wherein said plunging element serves to displace the said first and second bioadhesive components from said first and second lumen, into and through said first and second elongate tubes and said first and second chambers, and eject the mixed material from the distal portion of said nozzle element to said remote surgical site of interest.

2. The application assembly of claim 1, wherein the proximal end of said tubular applicator body is provided with a projecting flange.

3. The application assembly of claim 1, wherein the distal end of said elongate tubular section is provided with a cap.

4. The application assembly of claim 1, wherein said elongate tubular section is bendable.

5. The application assembly of claim 1, wherein the distal ends of said first and second distally extending elongate members are provided with sealing elements.

6. The application assembly of claim 1, wherein said nozzle component may be removably mounted to the distal end of said elongate tubular section.

7. The application assembly of claim 1, further comprising a plurality of gripping ridges disposed about the exterior periphery of said nozzle element.

8. The application assembly of claim 1, wherein the distal end of said nozzle element is tapered.

9. The applicator assembly of claim 1, wherein the proximal end of said nozzle element application the distal end of said elongate tubular section are provided with coordinating slots and protrusions that mate to form a locked fastener pair, to thereby secure the attachment of said nozzle to said

10. A sterile kit for delivering a two-part poly-synthetic bioadhesive to a remote surgical site of interest, said kit comprising:
i. the application assembly of claim 1, wherein said components (a)-(e) are disassembled from each other, the proximal end of said tubular applicator body is provided with a cap, and said first and second lumen are pre-loaded with first and second components of said two part poly-synethetic bioadhesive;
ii. One or more additional nozzle elements as recited in claim 1; and
iii. One or more additional mixing element as recited in claim 1.

11. The sterile kit of claim 10, wherein said first and second components of said two part poly-synethetic bioadhesive comprise discrete polyphenolic proteins that when mixed, said proteins cross-link to form said biocompatible poly-synthetic bioadhesive.

12. The sterile kit of claim 11, wherein said bioadhesive is immunogenic.

13. The sterile kit of claim 11, wherein said bioadhesive sets in 30 seconds or less.

14. The sterile kit of claim 11, wherein said bioadhesive is biodegradable.

15. The sterile kit of claim 11, wherein said bioadhesive is derived from the sandcastle worm.

16. A method for the minimally invasive repair of a tissue tear at a remote site in the body of a patient, said method comprising the step of:
i. providing the application assembly of claim 1, wherein said first and second lumen are pre-loaded with first and second components of a two part poly-synethetic bioadhesive;
ii. assembling plunging element (e) to tubular applicator body (a) and slidably inserting the first and second distally extending elongate members of said plunging element a first distance into the respective first and second lumen so as to displace said first and second bioadhesive components from said first and second lumen, into said first and second elongate tubes, until said bioadhesive components appear at the distal ends of said first and second elongate tubes;
iii. assembling mixing element (c) and nozzle element (d) to the distal end of said elongate tubular section to form an assembled application device;
iv. introducing said assembled application device into the patient and positioning said nozzle element adjacent to said remote tissue tear;
v. further driving said plunging element a second distance into the respective first and second lumen so as to displace said first and second bioadhesive components from said first and second elongate tubes, into and through said first and second chambers and into the distal portion of said nozzle element; and
vi. introducing the mixed bioadhesive into the tissue tear and allowing the adhesive to set.

17. The method of claim 16, wherein said first and second components of said two part poly-synethetic bioadhesive comprise discrete polyphenolic proteins that when mixed, said proteins cross-link to form said biocompatible poly-synthetic bioadhesive.

18. The method of claim 16, wherein said tissue tear comprises a meniscal tear, a spinal disc herniation, or an annulus tear.

19. The method of claim 16, wherein the tissue at the remote tear site are held in a compressed position while the bioadhesive sets.

20. The method of claim 16, wherein said bioadhesive sets in 30 seconds or less.

21. The method of claim 16, further comprising the step of demounting mixing element (c) and nozzle element (d) from the distal end of said elongate tubular section and remounting new mixing and nozzle elements thereto.

22. The method of claim 16, wherein the distal end of elongate tubular section (b) is provided with a cap and the method further comprises the step of removing said cap prior step (ii).

23. The method of claim 16, further comprising step (vii) of using said nozzle element to compress said tissue tear during the time period in which said adhesive is setting.
